(19) United States
(12) Patent Application Publication
(10) Pub. No.: US 2026/0060660 A1
(43) Pub. Date: Mar. 5, 2026

(54) COMPOSITIONS AND METHODS FOR IMMUNOLOGICAL TESTING OF STOOL

(71) Applicant: GENEOSCOPY, INC., St. Louis, MO (US)

(72) Inventors: Erica BARNELL, St. Louis, MO (US); Andrew BARNELL, St. Louis, MO (US); Kimberly KRUSE, St. Louis, MO (US)

(21) Appl. No.: 19/314,697

(22) Filed: Aug. 29, 2025

Related U.S. Application Data
(60) Provisional application No. 63/689,280, filed on Aug. 30, 2024.

(51) Int. Cl. A61B 10/00; C12Q 1/6806; G01N 33/72

(52) U.S. Cl. CPC A61B 10/0038; C12Q 1/6806; G01N 33/721

(57) ABSTRACT

The present disclosure provides improved methods of processing a fecal sample by a human subject in which the fecal sample is collected at home and subsequently delivered to a medical diagnostics laboratory. As described herein, the means of collecting and processing the fecal sample can desirably reduce the number of steps undertaken by the human subject, including avoiding steps of removing or separating the fecal sample into multiple portions. Furthermore, the human subject does not have to perform an immunological test (e.g., a fecal immunochemical test (FIT) or an immunochemical fecal occult blood test (iFOBT)) at home.

### CROSS-REFERENCE TO RELATED APPLICATIONS

This application claims the benefit under 35 U.S.C. § 119(e) of U.S. Provisional Application Ser. No. 63/689,280, filed on Aug. 30, 2024, the entire disclosure of which is incorporated herein by reference.

### BACKGROUND AND SUMMARY

Gastrointestinal disorders, including gastrointestinal cancers, ulcerative colitis, irritable bowel syndrome, and Crohn's disease, affect millions of people worldwide. For instance, in the U.S. alone, approximately 60 to 70 million people are affected each year. Given the invasive nature of diagnostic methods such as colonoscopies, alternative non-invasive methods of diagnosing gastrointestinal disorders have been developed.

Certain non-invasive methods for diagnosing gastrointestinal disorders include those in which a human subject collects a stool sample at home and then transfers the sample to an external laboratory for process and evaluation. Although such at-home methods have demonstrated sensitivity and specificity to diagnose gastrointestinal disorders, improvements to the patient experience with respect to the procedures for collection and transport are constantly being evaluated. Such improvements can be pursued in order to increase patient compliance of known non-invasive methods and also to improve diagnostic efficacy.

Accordingly, the present disclosure provides novel methods of processing a fecal sample by a human subject in which the fecal sample is collected at home and subsequently delivered to a medical diagnostics laboratory. As described herein, the means of collecting and processing the fecal sample can desirably reduce the number of steps required by the human subject compared to the state of the art. For instance, according to the present disclosure, the human subject can avoid steps of removing or separating the fecal sample into multiple portions. Furthermore, the human subject can avoid the requirement to perform an immunological test (e.g., a fecal immunochemical test (FIT) or an immunochemical fecal occult blood test (iFOBT)) at home by simply instituting delivery of the sample to a laboratory for processing. Moreover, the means of collecting and processing the fecal sample as described herein can allow for parallel analyses to be performed on multiple types of analytes. The methods described herein can beneficially improve the patient experience while providing comparable or even improved efficacy in diagnosing gastrointestinal disorders.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a comparison of storage stability analysis for At-Home FIT results and In-Lab FIT results.

FIG. 2 shows a comparison of freeze-thaw stability analysis for At-Home FIT results and In-Lab FIT results.

FIG. 3 shows the concordance (positive vs. negative) of the At-Home FIT results and In-Lab FIT results using prospectively collected stool samples.

FIGS. 4A-4D show the results of various analytical evaluations. FIG. 4A displays a box plot showing in-lab FIT results for two stool pools (negative and positive) across four freeze-thaw cycles. FIG. 4B displays a box plot showing in-lab FIT results for two stool pools (negative and positive) across nine interfering substances. FIG. 4C displays a box plot showing in-lab FIT results for two stool pools (negative and positive) across five stool input volumes. FIG. 4D displays a bar plot showing concordance for low negative (LN) pool and percent coefficient of variation (% CV) for high negative (HN), low positive (LP), and high positive (HP) pools for the in-lab FIT when evaluated across multiple lots, days, users, and systems.

FIG. 5 shows FIT scores for the At-Home method and the In-Lab method as generated at six (6) timepoints (<6 hours, 24 hours, 48 hours, 72 hours, 96 hours, and 120 hours). FITs tested at various timepoints were assessed for concordance with the original samples type, which was characterized using the At-Home FIT method prior to aliquoting into tubes. The top two panels display results for at-home and in-lab method testing for aliquots in the negative pool. The bottom two panels display results for at-home and in-lab method testing for aliquots in the positive pool.

FIG. 6 shows FIT scores for the in-lab method after exposure to various buffer types. The left panel displays results using a mt-sRNA buffer. The right panel displays results using a comparative lysis buffer without stability properties.

### DETAILED DESCRIPTION

According to the various illustrative aspects described herein, the method steps for processing a fecal sample are simplified for efficiency and adherence by a human subject. As described herein, various method steps commonly required to be performed at home and/or by the human subject according to the state of the art are omitted in the present disclosure. Moreover, as described herein, a single liquid composition that can stabilize both fecal proteins and nucleic acid can be utilized according to the methods. As a result, a streamlined patient experience can be achieved.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) a portion of the fecal sample is not removed at home or ii) a portion of the fecal sample is not removed by the human subject. As used herein, “collecting” or “collection” of a fecal sample can involve defecation by the human subject into a suitable vessel. For instance, the human subject can defecate directly into a vessel such as a container, a bucket, a pail, or other similar vessel suitable for collection. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) a portion of the fecal sample is not removed to a separate container at home or ii) a portion of the fecal sample is not removed to a separate container by the human subject. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) a portion of the fecal sample is not removed to a separate scalable container at home or ii) a portion of the fecal sample is not removed to a separate scalable container by the human subject. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) the fecal sample is not divided into two or more portions at home or ii) the fecal sample is not divided into two or more portions by the human subject. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) the fecal sample is not divided into two or more portions by the human subject or ii) the fecal sample is not divided into two or more portions at home. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, wherein the method does not comprise contacting the fecal sample. As used herein, contacting can refer to physical manipulation of an object, for instance scooping, scraping, swabbing, or the like. Typically, the contacting of an object is performed using a different object (e.g., contacting a fecal sample could include use of a scoop, a scraper, a swab, or other objects). Typically, contacting of a fecal sample is performed for the purpose of removing or separating a portion of the sample into a separate container, and/or for mixing the sample, and/or for homogenizing the sample. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) the method does not comprise contacting the fecal sample with a fecal immunochemical test (FIT) or an immunochemical fecal occult blood test (iFOBT) at home or ii) the method does not comprise the human subject contacting the fecal sample with a fecal immunochemical test (FIT) or an immunochemical fecal occult blood test (iFOBT). In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) the method does not comprise contacting the fecal sample with a fecal immunochemical test (FIT) or an immunochemical fecal occult blood test (iFOBT) at home or ii) the method does not comprise the human subject contacting the fecal sample with a fecal immunochemical test (FIT) or an immunochemical fecal occult blood test (iFOBT). In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) the method does not comprise the human subject contacting the fecal sample with a fecal immunochemical test (FIT) or an immunochemical fecal occult blood test (iFOBT) or ii) the method does not comprise contacting the fecal sample with a fecal immunochemical test (FIT) or an immunochemical fecal occult blood test (iFOBT) at home. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) the method does not comprise performing a fecal immunochemical test (FIT) or an immunochemical fecal occult blood test (iFOBT) on the fecal sample at home or ii) the method does not comprise the human subject performing a fecal immunochemical test (FIT) or an immunochemical fecal occult blood test (iFOBT) on the fecal sample. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container, wherein the liquid composition prevents denaturation or degradation of fecal proteins in the fecal sample and wherein the liquid composition maintains integrity of nucleic acids in the fecal sample; and c) delivering the container containing the fecal sample and the liquid composition to a medical diagnostics laboratory, and wherein i) the method does not comprise performing a fecal immunochemical test (FIT) or an immunochemical fecal occult blood test (iFOBT) on the fecal sample by the human subject or ii) the method does not comprise performing a fecal immunochemical test (FIT) or an immunochemical fecal occult blood test (iFOBT) on the fecal sample at home. In an embodiment, the fecal sample is collected at home by the human subject via defecation directly into a container.

In an illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein a portion of the fecal sample is not removed.

In another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein a portion of the fecal sample is not removed to a separate container.

In yet another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein a portion of the fecal sample is not removed to a separate scalable container.

In another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein the fecal sample is not divided into two or more portions.

In yet another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein the fecal sample is not divided into two or more portions by the human subject.

In another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein the method does not comprise contacting the fecal sample with a fecal immunochemical test (FIT) or an immunochemical fecal occult blood test (iFOBT) at home.

In yet another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein the method does not comprise the human subject contacting the fecal sample with a fecal immunochemical test (FIT) or an immunochemical fecal occult blood test (iFOBT).

In another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein the method does not comprise performing a fecal immunochemical test (FIT) or an immunochemical fecal occult blood test (iFOBT) on the fecal sample at home.

In yet another illustrative aspect, a method of processing a fecal sample is provided. The method comprises the steps of a) collecting a fecal sample from a human subject, wherein the fecal sample is collected at home by the human subject; b) combining the fecal sample with a liquid composition in a container; and c) delivering the container containing the fecal sample and the liquid composition to a medical diagnostics laboratory, wherein the method does not comprise performing a fecal immunochemical test (FIT) or an immunochemical fecal occult blood test (iFOBT) on the fecal sample by the human subject.

In any of the illustrative aspects, the fecal sample is a whole fecal sample. In any of the illustrative aspects, the method is performed without freezing. In any of the illustrative aspects, the fecal sample is a freshly-collected fecal sample.

As used herein, “collecting” or “collection” of a fecal sample can involve defecation by the human subject into a suitable vessel. For instance, the human subject can defecate directly into a vessel such as a container, a bucket, a pail, or other similar vessel suitable for collection. In any of the illustrative aspects, the fecal sample is collected at home by the human subject via defecation directly into a container. In any of the illustrative aspects, the container is a scalable container.

In any of the illustrative aspects, the liquid composition prevents denaturation or degradation of fecal proteins in the fecal sample. As used herein, a “fecal protein” refers to any protein that is present in a fecal sample, for instance proteins produced by a cell in the gastrointestinal tract as well as proteins originating from other locations in the body such as the blood stream. In any of the illustrative aspects, a second liquid composition is not used, thus indicating that inclusion of the second liquid composition is not necessary to prevent denaturation or degradation of fecal proteins in the fecal sample.

In any of the illustrative aspects, the liquid composition maintains integrity of nucleic acids in the fecal sample. In any of the illustrative aspects, the liquid composition prevents denaturation or degradation of fecal proteins in the fecal sample and wherein the liquid composition maintains integrity of nucleic acids in the fecal sample. In any of the illustrative aspects, separation of the fecal sample prior to shipment to the medical diagnostics laboratory is not performed.

In any of the illustrative aspects, the liquid composition comprises a buffer. In any of the illustrative aspects, the buffer is a basic buffer. In any of the illustrative aspects, the basic buffer comprises a pH between about 7.2 to about 8.0. In any of the illustrative aspects, the buffer comprises an osmolality of about ˜308 mEq/L. In any of the illustrative aspects, the buffer is zwitterionic at a physiological pH.

In any of the illustrative aspects, the buffer is selected from the group consisting of Hanks balanced salt solution, Alsever's solution, Earle's balanced salt solution, Gey's balanced salt solution, Phosphate buffered saline, Puck's balanced salt solution, Ringer's balanced salt solution, Simm's balanced salt solution, TRIS-buffered saline, Tyrode's balanced salt solution, and any combination thereof.

In any of the illustrative aspects, the liquid composition comprises a surfactant. In any of the illustrative aspects, the surfactant is an ionic surfactant. In any of the illustrative aspects, the surfactant is a non-ionic surfactant. In any of the illustrative aspects, the surfactant comprises Tween-20. In any of the illustrative aspects, the surfactant comprises Triton-X-100.

In any of the illustrative aspects, the liquid composition comprises a ribonuclease inhibitor. In any of the illustrative aspects, the ribonuclease inhibitor is thiol-dependent. In any of the illustrative aspects, the ribonuclease inhibitor is selected from the group consisting of Protector RNase Inhibitor (Roche), RNasin® (Promega), SUPERase-In™ (Thermo Fisher Scientific), RNaseOUT™ (Thermo Fisher Scientific), ANTI-RNase, a Recombinant RNase Inhibitor, or a cloned RNase Inhibitor.

In any of the illustrative aspects, the fecal proteins comprise one or more of albumin, hemoglobin, calprotectin, lactoferrin, myeloperoxidase, α1-antitrypsin, lysozyme, elastase, transferrin, immunoglobulin A (IgA), immunoglobulin G (IgG), immunoglobulin M (IgM), pancreatic amylase, trypsin, chymotrypsin, mucin, defensin, or lipocalin-2. In any of the illustrative aspects, the fecal proteins comprise albumin. In any of the illustrative aspects, the fecal proteins comprise hemoglobin. In any of the illustrative aspects, the fecal proteins comprise calprotectin. In any of the illustrative aspects, the fecal proteins comprise lactoferrin. In any of the illustrative aspects, the fecal proteins comprise myeloperoxidase. In any of the illustrative aspects, the fecal proteins comprise α1-antitrypsin. In any of the illustrative aspects, the fecal proteins comprise lysozyme. In any of the illustrative aspects, the fecal proteins comprise elastase. In any of the illustrative aspects, the fecal proteins comprise transferrin. In any of the illustrative aspects, the fecal proteins comprise immunoglobulin A (IgA). In any of the illustrative aspects, the fecal proteins comprise immunoglobulin G (IgG). In any of the illustrative aspects, the fecal proteins comprise immunoglobulin M (IgM). In any of the illustrative aspects, the fecal proteins comprise pancreatic amylase. In any of the illustrative aspects, the fecal proteins comprise trypsin. In any of the illustrative aspects, the fecal proteins comprise chymotrypsin. In any of the illustrative aspects, the fecal proteins comprise mucin. In any of the illustrative aspects, the fecal proteins comprise defensin. In any of the illustrative aspects, the fecal proteins comprise lipocalin-2.

In an illustrative aspect, a method of processing a fecal sample is provided. The method comprises the steps of i) obtaining a fecal sample collected from a human subject according to any aspect described herein, and ii) analyzing the fecal sample for an amount of a fecal protein.

In an embodiment, the fecal sample is a whole fecal sample. In an embodiment, the fecal protein comprises one or more of albumin, hemoglobin, calprotectin, lactoferrin, myeloperoxidase, α1-antitrypsin, lysozyme, elastase, transferrin, immunoglobulin A (IgA), immunoglobulin G (IgG), immunoglobulin M (IgM), pancreatic amylase, trypsin, chymotrypsin, mucin, defensin, lipocalin-2, or any combination thereof. In an embodiment, the fecal protein is hemoglobin, the fecal protein is calprotectin, or both. In an embodiment, the fecal protein is albumin. In an embodiment, the fecal protein is hemoglobin. In an embodiment, the fecal protein is calprotectin. In an embodiment, the fecal protein is lactoferrin. In an embodiment, the fecal protein is myeloperoxidase. In an embodiment, the fecal protein is α1-antitrypsin. In an embodiment, the fecal protein is lysozyme. In an embodiment, the fecal protein is elastase. In an embodiment, the fecal protein is transferrin. In an embodiment, the fecal protein is immunoglobulin A (IgA). In an embodiment, the fecal protein is immunoglobulin G (IgG). In an embodiment, the fecal protein is immunoglobulin M (IgM). In an embodiment, the fecal protein is pancreatic amylase. In an embodiment, the fecal protein is trypsin. In an embodiment, the fecal protein is chymotrypsin. In an embodiment, the fecal protein is mucin. In an embodiment, the fecal protein is defensin. In an embodiment, the fecal protein is lipocalin-2.

In an embodiment, the fecal protein is hemoglobin. In an embodiment, the fecal protein is calprotectin. In an embodiment, analyzing the fecal sample of step ii) comprises testing for a concentration of hemoglobin in the fecal sample. In an embodiment, analyzing the fecal sample of step ii) comprises testing for a concentration of calprotectin in the fecal sample. In an embodiment, testing for the concentration of hemoglobin comprises immunochemical detection of hemoglobin. In an embodiment, testing for the concentration of hemoglobin comprises immunochemical detection of hemoglobin via a fecal immunochemical test (FIT) or an immunochemical fecal occult blood test (iFOBT).

In an embodiment, the fecal sample is considered positive for the presence of blood when the concentration of hemoglobin detected in the removed portion is at least 5 ng/ml. In an embodiment, the fecal sample is considered positive for the presence of blood when the concentration of hemoglobin detected in the removed portion is at least 10 ng/ml. In an embodiment, the fecal sample is considered positive for the presence of blood when the concentration of hemoglobin detected in the removed portion is at least 20 ng/ml. In an embodiment, the fecal sample is considered positive for the presence of blood when the concentration of hemoglobin detected in the removed portion is at least 50 ng/ml. In an embodiment, the fecal sample is considered positive for the presence of blood when the concentration of hemoglobin detected in the removed portion is at least 75 ng/ml. In an embodiment, the fecal sample is considered positive for the presence of blood when the concentration of hemoglobin detected in the removed portion is at least 100 ng/ml. In an embodiment, the fecal sample is considered positive for the presence of blood when the concentration of hemoglobin detected in the removed portion is at least 125 ng/ml. In an embodiment, the fecal sample is considered positive for the presence of blood when the concentration of hemoglobin detected in the removed portion is at least 150 ng/ml.

In an embodiment, analyzing the fecal sample of step ii) is performed in a laboratory. In an embodiment, analyzing the fecal sample of step ii) is performed in the medical diagnostics laboratory.

In an embodiment, analyzing the fecal sample of step ii) comprises contacting the fecal sample, for instance with a swab, a pipette, or any other means of removing a portion of the fecal sample. For such embodiments, the collection of the fecal sample can be performed at home by the human subject, but the fecal sample is not contacted by the human subject (e.g., at home). Subsequently, the fecal sample can be contacted in a laboratory (e.g., by laboratory personnel) to perform a step of analyzing the fecal sample. In an embodiment, contacting the fecal sample is performed using a swab. In an embodiment, contacting the fecal sample is performed using a pipette.

In an embodiment, analyzing the fecal sample of step ii) comprises removing a portion of the fecal sample, for instance with a swab, a pipette, or any other means of removing a portion of the fecal sample. For such embodiments, the collection of the fecal sample can be performed at home by the human subject, but a portion of the fecal sample is not removed by the human subject (e.g., at home). Subsequently, a portion of the fecal sample can be removed in a laboratory (e.g., by laboratory personnel) to perform a step of analyzing the fecal sample. In an embodiment, removing the portion of the fecal sample is performed using a swab. In an embodiment, removing the portion of the fecal sample is performed using a pipette.

In an embodiment, analyzing the fecal sample of step ii) is not performed at the home. In an embodiment, analyzing the fecal sample of step ii) is performed by a laboratory technician. In an embodiment, analyzing the fecal sample of step ii) is not performed by the human subject.

In an embodiment, the method further comprises a step of analyzing the fecal sample for an amount of a human nucleic acid. In an embodiment, the human nucleic acid is selected from the group consisting of DNA, methylated DNA, hypermethylated DNA, epigenetically modified DNA, RNA, and any combination thereof. In an embodiment, the human nucleic acid comprises DNA, or methylated DNA, or hypermethylated DNA, or epigenetically modified DNA. In an embodiment, the human nucleic acid comprises DNA. In an embodiment, the human nucleic acid comprises methylated DNA. In an embodiment, the human nucleic acid comprises hypermethylated DNA. In an embodiment, the human nucleic acid comprises epigenetically modified DNA. In an embodiment, the human nucleic acid comprises RNA. In an embodiment, the RNA is selected from the group consisting of seRNA, total RNA, mRNA, tRNA, rRNA, ncRNA, smRNA, miRNA, snoRNA, and any combination thereof.

In an embodiment, the analysis of the fecal sample comprises extraction of the human nucleic acid from the fecal sample. In an embodiment, the analysis of the fecal sample comprises measuring the level of expression of one or more stool-derived human nucleic acid biomarkers present in the fecal sample. In an embodiment, the method further comprises comparing the analysis of the fecal sample, wherein the comparison comprises measuring the level of expression of one or more stool-derived human nucleic acid biomarkers present in the fecal sample with a measured expression level of the one or more stool-derived human nucleic acid biomarkers in a control. In an embodiment, the method further comprises comparing the analysis of the fecal sample, wherein the comparison comprises measuring the level of or the presence of the fecal protein present in the fecal sample with a level or presence of fecal protein in a control, or a combination thereof. In an embodiment, the comparison provides a result that indicates that the human subject has colorectal neoplasia, or one or more other precancerous lesions, or both. In an embodiment, the comparison provides a result that indicates that the human subject has an inflammatory bowel disease (IBD), or ulcerative colitis (UC), or Crohn's disease (CD). In an embodiment, the comparison provides a result that indicates a status of IBD, UC, or CD in the human subject. In an embodiment, the comparison provides a result that indicates a feature of IBD, UC, or CD in the human subject. In an embodiment, the comparison provides a result that indicates the extent of IBD, UC, or CD in the human subject. In an embodiment, the comparison provides a result that indicates one or more therapy options for IBD, UC, or CD in the human subject.

In an embodiment, the method further comprises a step of administering a colonoscopy to the human subject. In an embodiment, the method further comprises a step of administering a treatment to the human subject. In an embodiment, the treatment is selected from the group consisting of surgery, chemotherapy, radiation therapy, targeted therapy, immunotherapy, and any combination thereof. In an embodiment, the treatment is surgery. In an embodiment, the treatment is chemotherapy. In an embodiment, the chemotherapy comprises administration of 5-fluorouracil, leucovorin, capecitabine, oxaliplatin, irinotecan, or any combination thereof. In an embodiment, the treatment is radiation therapy.

In an embodiment, the treatment is a targeted therapy. In an embodiment, the targeted therapy comprises administration of bevacizumab (anti-VEGF), ramuciramab (anti-VEGFR2), aflibercept, regorafenib, cetuximab (anti-EGFR), panitumumab, tripfluridine-tipiracil, encorafenib (BRAF inhibitor), binimetinib (MEK inhibitor), or any combination thereof. In an embodiment, the targeted therapy comprises administration of bevacizumab (anti-VEGF). In an embodiment, the targeted therapy comprises administration of ramuciramab (anti-VEGFR2). In an embodiment, the targeted therapy comprises administration of aflibercept. In an embodiment, the targeted therapy comprises administration of regorafenib. In an embodiment, the targeted therapy comprises administration of cetuximab (anti-EGFR). In an embodiment, the targeted therapy comprises administration of panitumumab. In an embodiment, the targeted therapy comprises administration of tripfluridine-tipiracil. In an embodiment, the targeted therapy comprises administration of encorafenib (BRAF inhibitor). In an embodiment, the targeted therapy comprises administration of binimetinib (MEK inhibitor).

In an embodiment, the treatment is an immunotherapy. In an embodiment, the immunotherapy is an immune checkpoint inhibitor. In an embodiment, the immunotherapy comprises pembrolizumab, nivolumab, ipilimumab, dostarlimab, or any combination thereof. In an embodiment, the immunotherapy is pembrolizumab (anti-PD-1). In an embodiment, the immunotherapy is nivolumab (anti-PD-1). In an embodiment, the immunotherapy is ipilimumab (anti-CTLA-4). In an embodiment, the immunotherapy is dostarlimab (anti-PD-1).

In any of the various aspects and embodiments described herein, the disclosures of one or more of the following publications may be applied in pertinent part: PCT Publication No. WO 2016/176446, PCT Publication No. WO 2018/081580, PCT Publication No. WO 2019/232483, PCT Publication No. WO 2024/163419, and U.S. Pat. No. 11,479,824. The disclosures of each of the above-noted publications are incorporated herein in their entirety. The following numbered embodiments are contemplated and are non-limiting:
1. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) a portion of the fecal sample is not removed at home or ii) a portion of the fecal sample is not removed by the human subject.
2. The method of clause 1, any other suitable clause, or any combination of suitable clauses, wherein the fecal sample is a whole fecal sample.
3. The method of clause 1, any other suitable clause, or any combination of suitable clauses, wherein the portion of the fecal sample is not removed at home.
4. The method of clause 1, any other suitable clause, or any combination of suitable clauses, wherein the portion of the fecal sample is not removed by the human subject.
5. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) a portion of the fecal sample is not removed to a separate container at home or ii) a portion of the fecal sample is not removed to a separate container by the human subject.
6. The method of clause 5, any other suitable clause, or any combination of suitable clauses, wherein the fecal sample is a whole fecal sample.
7. The method of clause 5, any other suitable clause, or any combination of suitable clauses, wherein the portion of the fecal sample is not removed to the separate container at home.
8. The method of clause 5, any other suitable clause, or any combination of suitable clauses, wherein the portion of the fecal sample is not removed to the separate container by the human subject.
9. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) a portion of the fecal sample is not removed to a separate sealable container at home or ii) a portion of the fecal sample is not removed to a separate sealable container by the human subject.
10. The method of clause 9, any other suitable clause, or any combination of suitable clauses, wherein the fecal sample is a whole fecal sample.
11. The method of clause 9, any other suitable clause, or any combination of suitable clauses, wherein the portion of the fecal sample is not removed to the separate sealable container at home.
12. The method of clause 9, any other suitable clause, or any combination of suitable clauses, wherein the portion of the fecal sample is not removed to the separate sealable container by the human subject.
13. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) the fecal sample is not divided into two or more portions at home or ii) the fecal sample is not divided into two or more portions by the human subject.
14. The method of clause 13, any other suitable clause, or any combination of suitable clauses, wherein the fecal sample is a whole fecal sample.
15. The method of clause 13, any other suitable clause, or any combination of suitable clauses, wherein the fecal sample is not divided into two or more portions at home.
16. The method of clause 13, any other suitable clause, or any combination of suitable clauses, wherein the fecal sample is not divided into two or more portions by the human subject.
17. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) the fecal sample is not divided into two or more portions by the human subject or ii) the fecal sample is not divided into two or more portions at home.
18. The method of clause 17, any other suitable clause, or any combination of suitable clauses, wherein the fecal sample is a whole fecal sample.
19. The method of clause 17, any other suitable clause, or any combination of suitable clauses, wherein the fecal sample is not divided into two or more portions by the human subject.
20. The method of clause 17, any other suitable clause, or any combination of suitable clauses, wherein the fecal sample is not divided into two or more portions at home.
21. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory,
wherein the method does not comprise contacting the fecal sample.
22. The method of clause 21, any other suitable clause, or any combination of suitable clauses, wherein the method does not comprise contacting the fecal sample by the human subject.
23. The method of clause 21, any other suitable clause, or any combination of suitable clauses, wherein the method does not comprise contacting the fecal sample with a fecal immunochemical test (FIT).
24. The method of clause 21, any other suitable clause, or any combination of suitable clauses, wherein the method does not comprise contacting the fecal sample with an immunochemical fecal occult blood test (iFOBT).
25. The method of clause 21, any other suitable clause, or any combination of suitable clauses, wherein the method does not comprise contacting the fecal sample with a fecal immunochemical test (FIT) at home.
26. The method of clause 21, any other suitable clause, or any combination of suitable clauses, wherein the method does not comprise contacting the fecal sample with an immunochemical fecal occult blood test (iFOBT) at home.
27. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) the method does not comprise contacting the fecal sample with a fecal immunochemical test (FIT) or an immunochemical fecal occult blood test (iFOBT) at home or ii) the method does not comprise the human subject contacting the fecal sample with a fecal immunochemical test (FIT) or an immunochemical fecal occult blood test (iFOBT).
28. The method of clause 27, any other suitable clause, or any combination of suitable clauses, wherein the method does not comprise contacting the fecal sample with the FIT at home.
29. The method of clause 27, any other suitable clause, or any combination of suitable clauses, wherein the method does not comprise contacting the fecal sample with the iFOBT at home.
30. The method of clause 27, any other suitable clause, or any combination of suitable clauses, wherein the method does not comprise the human subject contacting the fecal sample with the FIT.
31. The method of clause 27, any other suitable clause, or any combination of suitable clauses, wherein the method does not comprise the human subject contacting the fecal sample with the iFOBT.
32. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) the method does not comprise the human subject contacting the fecal sample with a fecal immunochemical test (FIT) or an immunochemical fecal occult blood test (iFOBT) or ii) the method does not comprise contacting the fecal sample with a fecal immunochemical test (FIT) or an immunochemical fecal occult blood test (iFOBT) at home.
33. The method of clause 32, any other suitable clause, or any combination of suitable clauses, wherein the method does not comprise the human subject contacting the fecal sample with the FIT.
34. The method of clause 32, any other suitable clause, or any combination of suitable clauses, wherein the method does not comprise the human subject contacting the fecal sample with the iFOBT.
35. The method of clause 32, any other suitable clause, or any combination of suitable clauses, wherein the method does not comprise contacting the fecal sample with the FIT at home.
36. The method of clause 32, any other suitable clause, or any combination of suitable clauses, wherein the method does not comprise contacting the fecal sample with the iFOBT at home.
37. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) the method does not comprise performing a fecal immunochemical test (FIT) or an immunochemical fecal occult blood test (iFOBT) on the fecal sample at home or ii) the method does not comprise the human subject performing a fecal immunochemical test (FIT) or an immunochemical fecal occult blood test (iFOBT) on the fecal sample.
38. The method of clause 37, any other suitable clause, or any combination of suitable clauses, wherein the method does not comprise performing the FIT on the fecal sample at home.
39. The method of clause 37, any other suitable clause, or any combination of suitable clauses, wherein the method does not comprise performing the iFOBT on the fecal sample at home.
40. The method of clause 37, any other suitable clause, or any combination of suitable clauses, wherein the method does not comprise the human subject performing the FIT on the fecal sample.
41. The method of clause 37, any other suitable clause, or any combination of suitable clauses, wherein the method does not comprise the human subject performing the iFOBT on the fecal sample.
42. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) the method does not comprise performing a fecal immunochemical test (FIT) or an immunochemical fecal occult blood test (iFOBT) on the fecal sample by the human subject or ii) the method does not comprise performing a fecal immunochemical test (FIT) or an immunochemical fecal occult blood test (iFOBT) on the fecal sample at home.
43. The method of clause 42, any other suitable clause, or any combination of suitable clauses, wherein the method does not comprise performing the FIT on the fecal sample by the human subject.
44. The method of clause 42, any other suitable clause, or any combination of suitable clauses, wherein the method does not comprise performing the iFOBT on the fecal sample by the human subject.
45. The method of clause 42, any other suitable clause, or any combination of suitable clauses, wherein the method does not comprise performing the FIT on the fecal sample at home.
46. The method of clause 42, any other suitable clause, or any combination of suitable clauses, wherein the method does not comprise performing the iFOBT on the fecal sample at home.
47. The method of any one of clauses 1 to 46, any other suitable clause, or any combination of suitable clauses, wherein the fecal sample is a whole fecal sample.
48. The method of any one of clauses 1 to 46, any other suitable clause, or any combination of suitable clauses, wherein the method is performed without freezing.
49. The method of any one of clauses 1 to 46, any other suitable clause, or any combination of suitable clauses, wherein the fecal sample is a freshly-collected fecal sample.
50. The method of any one of clauses 1 to 46, any other suitable clause, or any combination of suitable clauses, wherein the fecal sample is collected at home by the human subject via defecation directly into the container.
51. The method of any one of clauses 1 to 46, any other suitable clause, or any combination of suitable clauses, wherein the container is a sealable container.
52. The method of any one of clauses 1 to 46, any other suitable clause, or any combination of suitable clauses, wherein the liquid composition prevents denaturation or degradation of fecal proteins in the fecal sample.
53. The method of clause 52, any other suitable clause, or any combination of suitable clauses, wherein a second liquid composition is not used.
54. The method of any one of clauses 1 to 46, any other suitable clause, or any combination of suitable clauses, wherein the liquid composition maintains integrity of DNA in the fecal sample.
55. The method of clause 54, any other suitable clause, or any combination of suitable clauses, wherein a second liquid composition is not used.
56. The method of any one of clauses 1 to 46, any other suitable clause, or any combination of suitable clauses, wherein the liquid composition maintains integrity of RNA (e.g., seRNA, total RNA, mRNA, tRNA, rRNA, ncRNA, smRNA, miRNA, snoRNA, and any combination thereof) in the fecal sample.
57. The method of clause 56, any other suitable clause, or any combination of suitable clauses, wherein a second liquid composition is not used.
58. The method of any one of clauses 1 to 46, any other suitable clause, or any combination of suitable clauses, wherein the liquid composition comprises a buffer.
59. The method of clause 58, any other suitable clause, or any combination of suitable clauses, wherein the buffer is a basic buffer.
60. The method of clause 59, any other suitable clause, or any combination of suitable clauses, wherein the basic buffer comprises a pH between about 7.2 to about 8.0.
61. The method of clause 58, any other suitable clause, or any combination of suitable clauses, wherein the buffer comprises an osmolality of about ˜308 mEq/L.
62. The method of clause 58, any other suitable clause, or any combination of suitable clauses, wherein the buffer is zwitterionic at a physiological pH.
63. The method of clause 58, any other suitable clause, or any combination of suitable clauses, wherein the buffer is selected from the group consisting of Hanks balanced salt solution, Alsever's solution, Earle's balanced salt solution, Gey's balanced salt solution, phosphate buffered saline, Puck's balanced salt solution, Ringer's balanced salt solution, Simm's balanced salt solution, TRIS-buffered saline, Tyrode's balanced salt solution, and any combination thereof.
64. The method of clause 58, any other suitable clause, or any combination of suitable clauses, wherein the buffer comprises Hanks balanced salt solution.
65. The method of clause 58, any other suitable clause, or any combination of suitable clauses, wherein the buffer comprises Alsever's solution.
66. The method of clause 58, any other suitable clause, or any combination of suitable clauses, wherein the buffer comprises Earle's balanced salt solution.
67. The method of clause 58, any other suitable clause, or any combination of suitable clauses, wherein the buffer comprises Gey's balanced salt solution.
68. The method of clause 58, any other suitable clause, or any combination of suitable clauses, wherein the buffer comprises phosphate buffered saline.
69. The method of clause 58, any other suitable clause, or any combination of suitable clauses, wherein the buffer comprises Puck's balanced salt solution.
70. The method of clause 58, any other suitable clause, or any combination of suitable clauses, wherein the buffer comprises Ringer's balanced salt solution.
71. The method of clause 58, any other suitable clause, or any combination of suitable clauses, wherein the buffer comprises Simm's balanced salt solution.
72. The method of clause 58, any other suitable clause, or any combination of suitable clauses, wherein the buffer comprises TRIS-buffered saline.
73. The method of clause 58, any other suitable clause, or any combination of suitable clauses, wherein the buffer comprises Tyrode's balanced salt solution.
74 The method of any one of clauses 1 to 46, any other suitable clause, or any combination of suitable clauses, wherein the liquid composition comprises a surfactant.
75. The method of clause 74, any other suitable clause, or any combination of suitable clauses, wherein the surfactant is an ionic surfactant.
76. The method of clause 74, any other suitable clause, or any combination of suitable clauses, wherein the surfactant is a non-ionic surfactant.
77. The method of clause 74, any other suitable clause, or any combination of suitable clauses, wherein the surfactant comprises Tween-20.
78. The method of clause 74, any other suitable clause, or any combination of suitable clauses, wherein the surfactant comprises Triton-X-100.
79. The method of any one of clauses 1 to 46, any other suitable clause, or any combination of suitable clauses, wherein the liquid composition comprises a ribonuclease inhibitor.
80. The method of clause 79, any other suitable clause, or any combination of suitable clauses, wherein the ribonuclease inhibitor is thiol-dependent.
81. The method of clause 79, any other suitable clause, or any combination of suitable clauses, wherein the ribonuclease inhibitor is selected from the group consisting of Protector RNase Inhibitor (Roche), RNasin® (Promega), SUPERase-In™ (Thermo Fisher Scientific), RNaseOUT™ (Thermo Fisher Scientific), ANTI-RNase, a Recombinant RNase Inhibitor, or a cloned RNase Inhibitor.
82. The method of clause 79, any other suitable clause, or any combination of suitable clauses, wherein the ribonuclease inhibitor comprises Protector RNase Inhibitor.
83. The method of clause 79, any other suitable clause, or any combination of suitable clauses, wherein the ribonuclease inhibitor comprises RNasin®.
84. The method of clause 79, any other suitable clause, or any combination of suitable clauses, wherein the ribonuclease inhibitor comprises SUPERase-In™ (Thermo Fisher Scientific),
85. The method of clause 79, any other suitable clause, or any combination of suitable clauses, wherein the ribonuclease inhibitor comprises RNaseOUT™
86. The method of clause 79, any other suitable clause, or any combination of suitable clauses, wherein the ribonuclease inhibitor comprises ANTI-RNase.
87. The method of clause 79, any other suitable clause, or any combination of suitable clauses, wherein the ribonuclease inhibitor comprises a recombinant RNase Inhibitor.
88. The method of clause 79, any other suitable clause, or any combination of suitable clauses, wherein the ribonuclease inhibitor comprises a cloned RNase Inhibitor.
89. The method of any one of clauses 1 to 46, any other suitable clause, or any combination of suitable clauses, wherein the fecal proteins comprise one or more of albumin, hemoglobin, calprotectin, lactoferrin, myeloperoxidase, α1-antitrypsin, lysozyme, elastase, transferrin, immunoglobulin A (IgA), immunoglobulin G (IgG), immunoglobulin M (IgM), pancreatic amylase, trypsin, chymotrypsin, mucin, defensin, or lipocalin-2.
90. The method of any one of clauses 1 to 46, any other suitable clause, or any combination of suitable clauses, wherein the fecal proteins comprise albumin.
91. The method of any one of clauses 1 to 46, any other suitable clause, or any combination of suitable clauses, wherein the fecal proteins comprise hemoglobin.
92. The method of any one of clauses 1 to 46, any other suitable clause, or any combination of suitable clauses, wherein the fecal proteins comprise calprotectin.
93. The method of any one of clauses 1 to 46, any other suitable clause, or any combination of suitable clauses, wherein the fecal proteins comprise lactoferrin.
94. The method of any one of clauses 1 to 46, any other suitable clause, or any combination of suitable clauses, wherein the fecal proteins comprise myeloperoxidase.
95. The method of any one of clauses 1 to 46, any other suitable clause, or any combination of suitable clauses, wherein the fecal proteins comprise α1-antitrypsin.
96. The method of any one of clauses 1 to 46, any other suitable clause, or any combination of suitable clauses, wherein the fecal proteins comprise lysozyme.
97. The method of any one of clauses 1 to 46, any other suitable clause, or any combination of suitable clauses, wherein the fecal proteins comprise elastase.
98. The method of any one of clauses 1 to 46, any other suitable clause, or any combination of suitable clauses, wherein the fecal proteins comprise transferrin.
99. The method of any one of clauses 1 to 46, any other suitable clause, or any combination of suitable clauses, wherein the fecal proteins comprise immunoglobulin A (IgA).
100. The method of any one of clauses 1 to 46, any other suitable clause, or any combination of suitable clauses, wherein the fecal proteins comprise immunoglobulin G (IgG).
101. The method of any one of clauses 1 to 46, any other suitable clause, or any combination of suitable clauses, wherein the fecal proteins comprise immunoglobulin M (IgM).
102. The method of any one of clauses 1 to 46, any other suitable clause, or any combination of suitable clauses, wherein the fecal proteins comprise pancreatic amylase.
103. The method of any one of clauses 1 to 46, any other suitable clause, or any combination of suitable clauses, wherein the fecal proteins comprise trypsin.
104. The method of any one of clauses 1 to 46, any other suitable clause, or any combination of suitable clauses, wherein the fecal proteins comprise chymotrypsin.
105. The method of any one of clauses 1 to 46, any other suitable clause, or any combination of suitable clauses, wherein the fecal proteins comprise mucin.
106. The method of any one of clauses 1 to 46, any other suitable clause, or any combination of suitable clauses, wherein the fecal proteins comprise defensin.
107. The method of any one of clauses 1 to 46, any other suitable clause, or any combination of suitable clauses, wherein the fecal proteins comprise lipocalin-2.
108. A method of processing a fecal sample, the method comprising:
i) receiving a fecal sample in a liquid composition that prevents denaturation or degradation of one or more fecal proteins in the fecal sample, wherein the liquid composition maintains integrity of one or more human nucleic acids in the fecal sample, and
ii) analyzing the fecal sample for an amount of the fecal protein, and
iii) analyzing the fecal sample for an amount of the human nucleic acid.
109. The method of clause 108, any other suitable clause, or any combination of suitable clauses, wherein the fecal sample is a whole fecal sample.
110. The method of clause 108, any other suitable clause, or any combination of suitable clauses, wherein the fecal protein comprises albumin, hemoglobin, calprotectin, lactoferrin, myeloperoxidase, α1-antitrypsin, lysozyme, elastase, transferrin, immunoglobulin A (IgA), immunoglobulin G (IgG), immunoglobulin M (IgM), pancreatic amylase, trypsin, chymotrypsin, mucin, defensin, lipocalin-2, or any combination thereof.
111. The method of clause 108, any other suitable clause, or any combination of suitable clauses, wherein the fecal protein is hemoglobin, the fecal protein is calprotectin, or both.
112. The method of clause 108, any other suitable clause, or any combination of suitable clauses, wherein the fecal protein is albumin.
113. The method of clause 108, any other suitable clause, or any combination of suitable clauses, wherein the fecal protein is hemoglobin.
114. The method of clause 108, any other suitable clause, or any combination of suitable clauses, wherein the fecal protein is calprotectin.
115. The method of clause 108, any other suitable clause, or any combination of suitable clauses, wherein the fecal protein is lactoferrin.
116. The method of clause 108, any other suitable clause, or any combination of suitable clauses, wherein the fecal protein is myeloperoxidase.
117. The method of clause 108, any other suitable clause, or any combination of suitable clauses, wherein the fecal protein is α1-antitrypsin.
118. The method of clause 108, any other suitable clause, or any combination of suitable clauses, wherein the fecal protein is lysozyme.
119. The method of clause 108, any other suitable clause, or any combination of suitable clauses, wherein the fecal protein is elastase.
120. The method of clause 108, any other suitable clause, or any combination of suitable clauses, wherein the fecal protein is transferrin.
121. The method of clause 108, any other suitable clause, or any combination of suitable clauses, wherein the fecal protein is immunoglobulin A (IgA).
122. The method of clause 108, any other suitable clause, or any combination of suitable clauses, wherein the fecal protein is immunoglobulin G (IgG).
123. The method of clause 108, any other suitable clause, or any combination of suitable clauses, wherein the fecal protein is immunoglobulin M (IgM).
124. The method of clause 108, any other suitable clause, or any combination of suitable clauses, wherein the fecal protein is pancreatic amylase.
125. The method of clause 108, any other suitable clause, or any combination of suitable clauses, wherein the fecal protein is trypsin.
126. The method of clause 108, any other suitable clause, or any combination of suitable clauses, wherein the fecal protein is chymotrypsin.
127. The method of clause 108, any other suitable clause, or any combination of suitable clauses, wherein the fecal protein is mucin.
128. The method of clause 108, any other suitable clause, or any combination of suitable clauses, wherein the fecal protein is defensin.
129. The method of clause 108, any other suitable clause, or any combination of suitable clauses, wherein the fecal protein is or lipocalin-2.
130. The method of clause 108, any other suitable clause, or any combination of suitable clauses, wherein step ii) comprises testing for a concentration of hemoglobin in the fecal sample, wherein step ii) comprises testing for a concentration of calprotectin in the fecal sample, or both.
131. The method of clause 130, any other suitable clause, or any combination of suitable clauses, wherein testing for the concentration of hemoglobin comprises immunochemical detection of hemoglobin.
132. The method of clause 130, any other suitable clause, or any combination of suitable clauses, wherein testing for the concentration of hemoglobin comprises immunochemical detection of hemoglobin via a fecal immunochemical test (FIT) or an immunochemical fecal occult blood test (iFOBT).
133. The method of clause 130, any other suitable clause, or any combination of suitable clauses, wherein the fecal sample is considered positive for the presence of blood when the concentration of hemoglobin detected in the removed portion is at least 50 ng/ml.
134. The method of clause 130, any other suitable clause, or any combination of suitable clauses, wherein the fecal sample is considered positive for the presence of blood when the concentration of hemoglobin detected in the removed portion is at least 75 ng/ml.
135. The method of clause 130, any other suitable clause, or any combination of suitable clauses, wherein the fecal sample is considered positive for the presence of blood when the concentration of hemoglobin detected in the removed portion is at least 100 ng/ml.
136. The method of clause 130, any other suitable clause, or any combination of suitable clauses, wherein the fecal sample is considered positive for the presence of blood when the concentration of hemoglobin detected in the removed portion is at least 125 ng/ml.
137. The method of clause 130, any other suitable clause, or any combination of suitable clauses, wherein the fecal sample is considered positive for the presence of blood when the concentration of hemoglobin detected in the removed portion is at least 150 ng/ml.
138. The method of clause 108, any other suitable clause, or any combination of suitable clauses, wherein step ii) comprises testing for a concentration of hemoglobin in the fecal sample.
139. The method of clause 108, any other suitable clause, or any combination of suitable clauses, wherein step ii) comprises testing for a concentration of calprotectin in the fecal sample.
140. The method of clause 108, any other suitable clause, or any combination of suitable clauses, wherein step ii) is performed in a laboratory.
141. The method of clause 108, any other suitable clause, or any combination of suitable clauses, wherein step ii) comprises contacting the fecal sample, removing a portion of the fecal sample, or both.
142. The method of clause 108, any other suitable clause, or any combination of suitable clauses, wherein step ii) comprises contacting the fecal sample.
143. The method of clause 142, any other suitable clause, or any combination of suitable clauses, wherein contacting the fecal sample is performed using a swab.
144. The method of clause 142, any other suitable clause, or any combination of suitable clauses, wherein contacting the fecal sample is performed using a pipette.
145. The method of clause 108, any other suitable clause, or any combination of suitable clauses, wherein step ii) comprises removing a portion of the fecal sample.
146. The method of clause 145, any other suitable clause, or any combination of suitable clauses, wherein removing the portion of the fecal sample is performed using a swab.
147. The method of clause 145, any other suitable clause, or any combination of suitable clauses, wherein removing the portion of the fecal sample is performed using a pipette.
148. The method of clause 108, any other suitable clause, or any combination of suitable clauses, wherein step ii) is performed in a medical diagnostics laboratory.
149. The method of clause 108, any other suitable clause, or any combination of suitable clauses, wherein step ii) is not performed at a home.
150. The method of clause 108, any other suitable clause, or any combination of suitable clauses, wherein step ii) is performed by a laboratory technician.
151. The method of clause 108, any other suitable clause, or any combination of suitable clauses, wherein step ii) is not performed by a human subject.
152. The method of clause 108, any other suitable clause, or any combination of suitable clauses, wherein step iii) is performed in a medical diagnostics laboratory.
153. The method of clause 108, any other suitable clause, or any combination of suitable clauses, wherein step iii) is not performed at a home.
154. The method of clause 108, any other suitable clause, or any combination of suitable clauses, wherein step iii) is performed by a laboratory technician.
155. The method of clause 108, any other suitable clause, or any combination of suitable clauses, wherein step iii) is not performed by a human subject.
156. The method of clause 108, any other suitable clause, or any combination of suitable clauses, wherein the human nucleic acid is selected from the group consisting of DNA, methylated DNA, hypermethylated DNA, epigenetically modified DNA, RNA, and any combination thereof.
157. The method of clause 108, any other suitable clause, or any combination of suitable clauses, wherein the human nucleic acid comprises DNA, or methylated DNA, or hypermethylated DNA, or epigenetically modified DNA.
158. The method of clause 108, any other suitable clause, or any combination of suitable clauses, wherein the human nucleic acid comprises DNA.
159. The method of clause 108, any other suitable clause, or any combination of suitable clauses, wherein the human nucleic acid comprises methylated DNA.
160. The method of clause 108, any other suitable clause, or any combination of suitable clauses, wherein the human nucleic acid comprises hypermethylated DNA.
161. The method of clause 108, any other suitable clause, or any combination of suitable clauses, wherein the human nucleic acid comprises epigenetically modified DNA.
162. The method of clause 108, any other suitable clause, or any combination of suitable clauses, wherein the human nucleic acid comprises RNA (e.g., seRNA, total RNA, mRNA, tRNA, rRNA, ncRNA, smRNA, miRNA, snoRNA, and any combination thereof).
163. The method of clause 108, any other suitable clause, or any combination of suitable clauses, wherein analysis of the fecal sample comprises extraction of the human nucleic acid from the fecal sample.
164. The method of clause 108, any other suitable clause, or any combination of suitable clauses, wherein analysis of the fecal sample comprises measuring the level of expression of one or more stool-derived human nucleic acid biomarkers present in the fecal sample.
165. The method of clause 142, any other suitable clause, or any combination of suitable clauses, further comprising comparing the analysis of the fecal sample comprises measuring the level of expression of one or more stool-derived human nucleic acid biomarkers present in the fecal sample with a measured expression level of the one or more stool-derived human nucleic acid biomarkers in a control, or wherein the comparison comprises measuring the level of or the presence of the fecal protein present in the fecal sample with a level or presence of fecal protein in a control, or a combination thereof.
166. The method of clause 165, any other suitable clause, or any combination of suitable clauses, wherein the comparison indicates that the human subject has colorectal neoplasia, or one or more other precancerous lesions, or an inflammatory bowel disease (IBD), or ulcerative colitis (UC), or Crohn's disease (CD), or any combination thereof.
167. The method of clause 165, any other suitable clause, or any combination of suitable clauses, wherein the comparison indicates that the human subject has colorectal neoplasia.
168. The method of clause 165, any other suitable clause, or any combination of suitable clauses, wherein the comparison indicates that the human subject has one or more other precancerous lesions.
169. The method of clause 165, any other suitable clause, or any combination of suitable clauses, wherein the comparison indicates that the human subject has an inflammatory bowel disease (IBD).
170. The method of clause 165, any other suitable clause, or any combination of suitable clauses, wherein the comparison indicates that the human subject has ulcerative colitis (UC).
171. The method of clause 165, any other suitable clause, or any combination of suitable clauses, wherein the comparison indicates that the human subject has Crohn's disease (CD).
172. The method of clause 165, any other suitable clause, or any combination of suitable clauses, further comprising a step of administering a colonoscopy to the human subject.
173. The method of clause 165, any other suitable clause, or any combination of suitable clauses, further comprising a step of administering a treatment to the human subject.
174. The method of clause 173, any other suitable clause, or any combination of suitable clauses, wherein the treatment is selected from the group consisting of surgery, chemotherapy, radiation therapy, targeted therapy, immunotherapy, and any combination thereof.
175. The method of clause 173, any other suitable clause, or any combination of suitable clauses, wherein the treatment is surgery.
176. The method of clause 173, any other suitable clause, or any combination of suitable clauses, wherein the treatment is chemotherapy.
177. The method of clause 176, any other suitable clause, or any combination of suitable clauses, wherein the chemotherapy comprises administration of 5-fluorouracil, leucovorin, capecitabine, oxaliplatin, irinotecan, or any combination thereof.
178. The method of clause 176, any other suitable clause, or any combination of suitable clauses, wherein the chemotherapy comprises administration of 5-fluorouracil.
179. The method of clause 176, any other suitable clause, or any combination of suitable clauses, wherein the chemotherapy comprises administration of leucovorin.
180. The method of clause 176, any other suitable clause, or any combination of suitable clauses, wherein the chemotherapy comprises administration of capecitabine.
181. The method of clause 176, any other suitable clause, or any combination of suitable clauses, wherein the chemotherapy comprises administration of oxaliplatin.
182. The method of clause 176, any other suitable clause, or any combination of suitable clauses, wherein the chemotherapy comprises administration of irinotecan.
183. The method of clause 173, any other suitable clause, or any combination of suitable clauses, wherein the treatment is radiation therapy.
184. The method of clause 173, any other suitable clause, or any combination of suitable clauses, wherein the treatment is a targeted therapy.
185. The method of clause 184, any other suitable clause, or any combination of suitable clauses, wherein the targeted therapy comprises administration of bevacizumab (anti-VEGF), ramuciramab (anti-VEGFR2), aflibercept, regorafenib, cetuximab (anti-EGFR), panitumumab, tripfluridine-tipiracil, encorafenib (BRAF inhibitor), binimetinib (MEK inhibitor), or any combination thereof.
186. The method of clause 184, any other suitable clause, or any combination of suitable clauses, wherein the targeted therapy comprises administration of bevacizumab (anti-VEGF).
187. The method of clause 184, any other suitable clause, or any combination of suitable clauses, wherein the targeted therapy comprises administration of ramuciramab (anti-VEGFR2).
188. The method of clause 184, any other suitable clause, or any combination of suitable clauses, wherein the targeted therapy comprises administration of aflibercept.
189. The method of clause 184, any other suitable clause, or any combination of suitable clauses, wherein the targeted therapy comprises administration of regorafenib.
190. The method of clause 184, any other suitable clause, or any combination of suitable clauses, wherein the targeted therapy comprises administration of cetuximab (anti-EGFR).
191. The method of clause 184, any other suitable clause, or any combination of suitable clauses, wherein the targeted therapy comprises administration of panitumumab.
192. The method of clause 184, any other suitable clause, or any combination of suitable clauses, wherein the targeted therapy comprises administration of tripfluridine-tipiracil.
193. The method of clause 184, any other suitable clause, or any combination of suitable clauses, wherein the targeted therapy comprises administration of encorafenib (BRAF inhibitor).
194. The method of clause 184, any other suitable clause, or any combination of suitable clauses, wherein the targeted therapy comprises administration of binimetinib (MEK inhibitor).
195. The method of clause 173, any other suitable clause, or any combination of suitable clauses, wherein the treatment is an immunotherapy.
196. The method of clause 195, any other suitable clause, or any combination of suitable clauses, wherein the immunotherapy is an immune checkpoint inhibitor.
197. The method of clause 195, any other suitable clause, or any combination of suitable clauses, wherein the immunotherapy comprises pembrolizumab, nivolumab, ipilimumab, dostarlimab, or any combination thereof.
198. The method of clause 195, any other suitable clause, or any combination of suitable clauses, wherein the immunotherapy is pembrolizumab (anti-PD-1).
199. The method of clause 195, any other suitable clause, or any combination of suitable clauses, wherein the immunotherapy is nivolumab (anti-PD-1).
200. The method of clause 195, any other suitable clause, or any combination of suitable clauses, wherein the immunotherapy is ipilimumab (anti-CTLA-4).
201. The method of clause 195, any other suitable clause, or any combination of suitable clauses, wherein the immunotherapy is dostarlimab (anti-PD-1).

### EXAMPLES

### Example 1

### Clinical Evaluation

A total of 1,079 stool samples were collected from subjects prior to receiving a screening colonoscopy. Collection kits were provided to subjects who deposited a stool sample and completed an At-Home FIT swab upon collection to prior adding a liquid composition comprising buffer and transporting the kit back to the laboratory. Samples were in transit with the liquid composition for up to 96 hours. After sample collection, subjects received a colonoscopy to define disease status, including colorectal cancer (CRC), advanced adenomas (AA), serrated precancerous lesions (SPL), or all other findings (NEG).

Once stool samples were received in the laboratory, the At-Home FIT swab was analyzed. Stool samples were processed immediately and/or stored at −80° C. for future processing. To assess the ability to perform the FIT swab and analysis in the laboratory (In-Lab FIT), a portion of the processed stool sample was swabbed in the laboratory. The In-Lab FIT swab was analyzed. In-Lab FIT results were compared to At-Home FIT results and to colonoscopy results to assess concordance, sensitivity, and specificity.

Positivity rate for the At-Home method was 13% and positivity rate for the In-Lab method was 14%. Total concordance between the At-Home and In-Lab FIT assessment across all samples was 93% (1,004/1,079). There was no significant difference in the positivity rate between the two methods (Z-score p-value=0.66).

When evaluating discordant samples (n=75), the In-Lab FIT result was more closely associated with the colonoscopy results obtained for patients. For example, across the 75 discordant samples, 40% of the At-Home FIT results were aligned with the colonoscopy result whereas 60% of the In-Lab FIT results were aligned with the colonoscopy result. As shown in Table 1, relative to the At-Home FIT result, the In-Lab FIT result showed improvement in sensitivity for colorectal neoplasia (colorectal cancer [CRC] or advanced adenomas [AA]) and improvement in specificity for negative findings (NEG).

TABLE 1

| Category | At-Home | In-Lab |
|---|---|---|
| CRC (sensitivity) | 80% (15 / 20) | 80% (15 / 20) |
| AA (sensitivity) | 33% (76 / 231) | 38% (88 / 231) |
| SPL (sensitivity) | 13% (5 / 37) | 11% (4 / 37) |
| NEG (specificity) | 94% (747 / 791) | 95% (751 / 791) |

### Example 2

### Liquid Composition Analysis

Stool samples were obtained from 10 individuals that were known to be positive for hemoglobin based on the predetermined threshold. Each stool sample was split into replicates. Stool samples were swabbed with FIT swabs to assess the At-Home FIT method. Subsequently, aliquots were exposed to two different types of liquid compositions comprising buffers. One of the aliquots was exposed to the liquid compositions comprising buffer according to the present disclosure (“Disclosure”). In comparison, the second aliquot was exposed to a DNA preservative buffer (“Comparative”). Each aliquot sat in the liquid compositions for approximately 72 hours.

Samples were subsequently processed and assessed with the In-Lab FIT method. As shown in Table 2, concordance between the At-Home FIT and the In-Lab FIT was assessed. In total, 100% of samples that were exposed to the liquid compositions comprising buffer according to the present disclosure demonstrated concordance with the original sample type and only 20% of samples exposed to the DNA preservative buffer demonstrated concordance with the original sample type.

TABLE 2

| | Liquid | | | |
|---|---|---|---|---|
| | Composition | | | |
| Sample ID | Treatment | At-Home | In-Lab | Concordance |
| GENE_01_1 | Disclosure | Positive | Positive | Yes |
| GENE_01_2 | Disclosure | Positive | Positive | Yes |
| GENE_02_1 | Disclosure | Positive | Positive | Yes |
| GENE_02_2 | Disclosure | Positive | Positive | Yes |
| GENE_03_1 | Disclosure | Positive | Positive | Yes |
| GENE_03_2 | Disclosure | Positive | Positive | Yes |
| GENE_04_1 | Disclosure | Positive | Positive | Yes |
| GENE_04_2 | Disclosure | Positive | Positive | Yes |
| GENE_05_1 | Disclosure | Positive | Positive | Yes |
| GENE_05_2 | Disclosure | Positive | Positive | Yes |
| COMP_01_1 | Comparative | Positive | Positive | Yes |
| COMP_01_2 | Comparative | Positive | Negative | No |
| COMP_02_1 | Comparative | Positive | Negative | No |
| COMP_02_2 | Comparative | Positive | Negative | No |
| COMP_03_1 | Comparative | Positive | Positive | Yes |
| COMP_03_2 | Comparative | Positive | Negative | No |
| COMP_04_1 | Comparative | Positive | Negative | No |
| COMP_04_2 | Comparative | Positive | Negative | No |
| COMP_05_1 | Comparative | Positive | Negative | No |
| COMP_05_2 | Comparative | Positive | Negative | No |

### Example 3

### Storage Stability Analysis

Stool samples were obtained and aliquoted into two sample pools. Sample pools were spiked with peripheral hemoglobin to generate target concentrations above and below the FIT positivity threshold. Sample pools were split into 60 unique aliquots to represent patient samples. Aliquots were randomly allocated to a specific timepoint (<6 hours, 24 hours, 48 hours, 72 hours, 96 hours, 120 hours). All aliquots were queried with the At-Home FIT method by swabbing the stool sample with the OC-Auto FIT swab prior to adding liquid compositions comprising buffer.

Subsequently, all aliquots were exposed to liquid compositions comprising buffer according to the present disclosure for the associated time duration (<6 hours, 24 hours, 48 hours, 72 hours, 96 hours, 120 hours). After incubation, samples were assessed using the In-Lab FIT method.

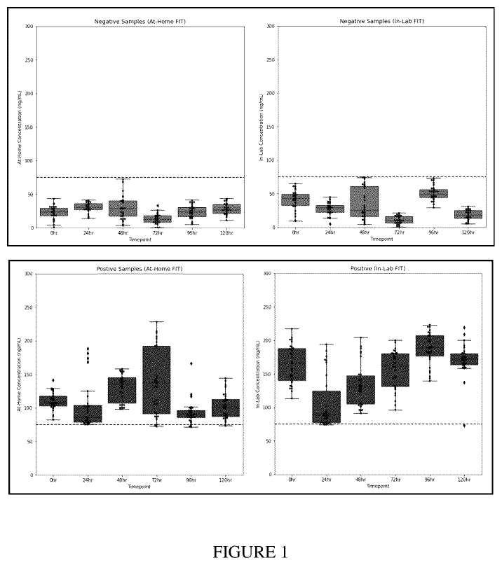

Both the At-Home FIT and the In-Lab FIT swabs were read at the same time using the OC-Auto Micro 80 FIT reader to assess in-transit stability. The At-Home FIT result was compared to the In-Lab FIT result (see FIG. 1). For aliquots generated from the negative stool pool, 100% of At-Home FIT replicates and 100% of In-Lab FIT replicates generated a negative result. For aliquots generated from the positive stool pool, 97% of At-Home FIT replicates and 98% of In-Lab FIT replicates generated a positive result.

### Example 4

### Freeze-Thaw Stability Analysis

Stool samples were obtained and aliquoted into two sample pools. Sample pools were spiked with peripheral hemoglobin to generate target concentrations above and below the FIT positivity threshold. Sample pools were assessed with the At-Home FIT method by swabbing the stool sample with the OC-Auto FIT swab. At-Home FIT swabs were tested using the OC-Auto Micro 80 FIT reader. Sample pools were then split into 20 unique aliquots to represent patient samples. Aliquots were randomly allocated to a specific freeze thaw (FT) cycle (0 FT cycles, 1 FT cycle, 2 FT cycles, or 3 FT cycles), which required thawing stool samples for more than 2 hours and freezing at <−60° C. for more than 24 hours. After the associated FT cycle, aliquots were assessed using the In-Lab FIT method.

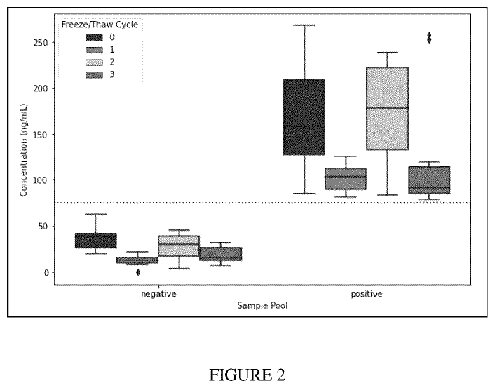

The In-Lab FIT swabs were read using the OC-Auto Micro 80 FIT reader. The At-Home FIT result was compared to the In-Lab FIT result (see FIG. 2). For aliquots generated from the negative stool pool, 100% of In-Lab FIT replicates generated a negative result. For aliquots generated from the positive stool pool, 100% of In-Lab FIT replicates generated a positive result.

### Example 5

### Prospective Clinical Stool Analysis

Stool samples were prospectively obtained from 51 human subjects using a collection kit. Subjects were instructed to generate a stool sample, complete the At-Home FIT swab and pour the liquid composition comprising buffer according to the present disclosure onto the stool sample prior to sending the kit back to the laboratory within 96 hours of processing.

In the laboratory, technicians evaluated the At-Home FIT swab completed by the subject using the OC-Auto Micro 80 FIT reader. Technicians also assessed the remaining stool portion using the In-Lab FIT swab method according to the present disclosure. The In-Lab FIT swabs were read using the OC-Auto Micro 80 FIT reader. The At-Home FIT result was compared to the In-Lab FIT result.

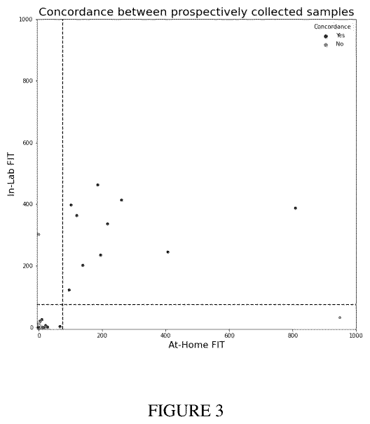

There were 13 positive samples (25%) and 38 negative samples (75%). Concordance was assessed between the At-Home FIT swab and the In-Lab FIT swab. As shown in FIG. 3, concordance between the two processes was 96% (49/51 samples).

### Example 6

### Further Analytical Analyses

The instant example provides additional analytical analyses the in-lab FIT methods described herein. Stool samples were collected following shipment of mt-sRNA collection kits at ambient temperature to subjects' residence. Subjects collected stool samples using instructions that included those to complete an at-home FIT in order to obtain a baseline hemoglobin concentration for stool samples. Subjects then shipped completed collection kits to the laboratory at ambient temperature, allowing for up to 96 hours in transit.

In the laboratory, human stool samples were assessed for levels of hemoglobin and then mixed together to create sample pools targeting specific protein concentration characteristics. For the instant example, hemoglobin concentrations and/or ranges were used to create the exemplary sample pools. When needed, human peripheral blood from donor(s) were used to adjust the pool concentrations. Each sample pool was then divided into aliquots in order to generate eukaryotic cell fractions.

Analytical testing was performed by generating stool pools with known hemoglobin concentration values and exposing stool to various conditions prior to testing with in-lab FIT methods. Freeze-thaw stability was assessed by subjecting stool pools to 0 to 3 freeze-thaw cycles and evaluating concordance of in-lab FIT results with the original sample classification (positive or negative). Interfering substances were evaluated by incubating stool in mt-sRNA buffer with common dietary components (e.g., red radish, broccoli, cauliflower, horseradish, turnip, cantaloupe, beef, pork, and parsnip). Stool input volume was tested by combining 50 to 250 grams of stool with 250 mL of stabilization buffer prior to in-lab FIT analysis. Precision was measured as the coefficient of variation (% CV) or concordance across three FIT lots, two laboratory users, three days, and two instruments. In-transit stability was evaluated by incubating stool in mt-sRNA buffer across six timepoints (0 to 96 hours).

Both the at-home and in-lab FITs were assessed using the OC Auto Micro 80 Analyzer (510(k), k041408). The same cutoff threshold (75 ng/mL) (positive versus negative) was used for both the at-home and in-lab FIT.

### Freeze Thaw Stability

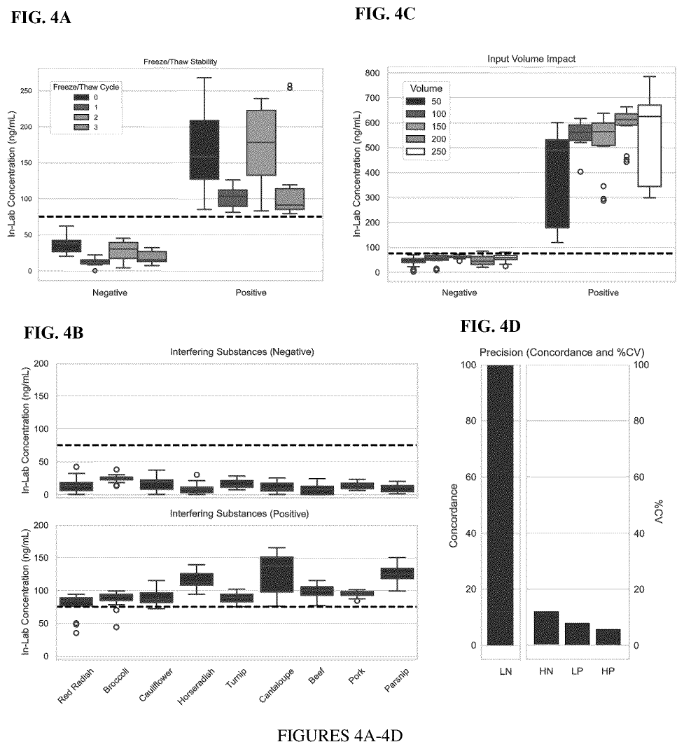

To assess freeze thaw stability, negative and positive stool pools were assessed with the at-home FIT methods, aliquoted into replicates, and then exposed to up to 3 freeze thaw cycles (24 hours at −60° C. and 2 hours at ambient temperature) prior to assessment with the in-lab FIT methods. When comparing at-home FIT results to in-lab FIT results, all replicates (n=30 replicates per freeze-thaw scenario) demonstrated concordance with the original sample classification (positive or negative). Table 3 and FIG. 4A display the results of these analyses.

### Interfering Substances

To assess interfering substances, negative and positive stool pools were assessed with the at-home FIT, aliquoted into replicates, and then exposed to various dietary substances (e.g., red radish, broccoli, cauliflower, horseradish, turnip, cantaloupe, beef, pork, and parsnip). Across all nine dietary substances (n=30 replicates per substance), ≥90% concordance was observed with the original sample classification (positive or negative). Table 3 and FIG. 4B display the results of these analyses.

### Stool Input

To evaluate fluctuation in stool input volumes with varied dilution factors when using the in-lab FIT methods, various volumes of stool (50 g, 100 g, 150 g, 200 g, and 250 g) were assessed with the at-home FIT methods prior to adding 250 mL stabilization buffer, and subsequently assessed with the in-lab FIT methods. Overall concordance across all replicates (n ≥30 replicates stool input volume), ≥96% concordance was observed with the original sample classification (positive or negative). Table 3 and FIG. 4C display the results of these analyses.

### Precision

Precision was used to assess total variance within the test system as related to days, operators, reagent lots, and instruments. Assessments were performed for four exemplary FIT concentration levels (e.g., low negative, high negative, low positive and high positive). Pools of stool material were generated to target the following exemplary concentrations: 10 ng/mL, 50 ng/mL, 100 ng/mL, and 140 ng/mL as determined using the at-home FIT method. Subsequently, replicates were assessed using the in-lab FIT methods across three unique days, two unique operators, three reagent lots, and two unique instruments. For high negative, low positive, and high positive stool pools (n=108 replicates per pool), the percentage of CV was 14.3%, 9.5%, and 6.8%, respectively. The low negative pool (n=108 replicates) showed 100% concordance. Table 3 and FIG. 4D display the results of these analyses.

### In-Transit Stability

The in-lab FIT method was performed after sample collection, buffer stabilization, and sample transit at ambient temperature for up to 96 hours. For the instant example, stability was assessed at six different incubation timepoints (<6, 24, 48, 72, 96, and 120 hours). Pools of stool material were assessed with the at-home FIT prior to aliquoting and exposure to stabilization buffer for the required timepoint. Subsequently, replicates were assessed using the in-lab FIT methods.

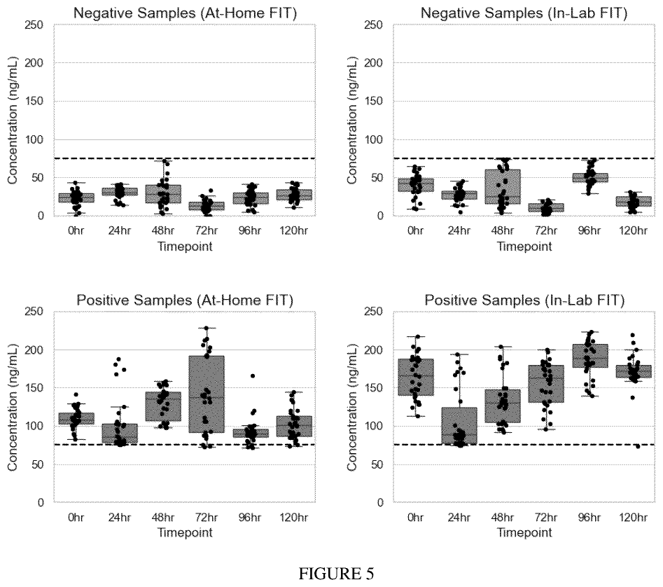

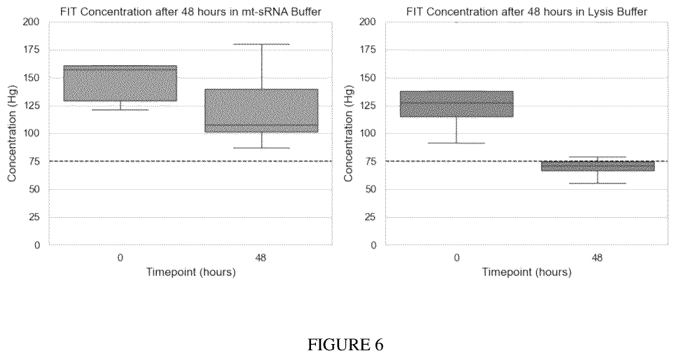

Across all replicates (n=60 replicates per timepoint), ≥97% concordance was observed with the original sample classification (positive or negative) (see Table 3 and FIG. 5). In contrast, exposure to a buffer that does not preferentially preserve cells in stool samples demonstrated a time-dependent decline in concordance for positive samples on the at-home FIT (see FIG. 6).

TABLE 3
Summary of analytical validation results for the in-
lab FIT assessment. Acceptance criteria required ≥85%
concordance or <15% coefficient of variation (CV)
across all samples within a given experiment.

| Study | Sample Size | Result |
|---|---|---|
| Freeze-Thaw | | |
| 0 freeze-thaw cycles | n = 30 | 100% concordance |
| 1 freeze-thaw cycles | n = 30 | 100% concordance |
| 2 freeze-thaw cycles | n = 30 | 100% concordance |
| 3 freeze-thaw cycles | n = 30 | 100% concordance |
| Interfering Substances | | |
| Red radish | n = 30 | 90% concordance |
| Broccoli | n = 30 | 93% concordance |
| Cauliflower | n = 30 | 97% concordance |
| Horseradish | n = 30 | 100% concordance |
| Turnip | n = 30 | 100% concordance |
| Cantaloupe | n = 30 | 100% concordance |
| Beef | n = 30 | 100% concordance |
| Pork | n = 30 | 100% concordance |
| Parsnip | n = 30 | 100% concordance |
| Stool Input | | |
| 50 g stool / 250 ml buffer | n = 48 | 100% concordance |
| 100 g stool / 250 ml buffer | n = 30 | 100% concordance |
| 150 g stool / 250 ml buffer | n = 30 | 97% concordance |
| 200 g stool / 250 ml buffer | n = 30 | 97% concordance |
| 250 g stool / 250 ml buffer | n = 48 | 96% concordance |
| Precision | | |
| Low Negative (Concordance) | n = 108 | 100% concordance |
| High Negative (% CV) | n = 108 | 14.3% CV |
| Low Positive (% CV) | n = 108 | 9.5% CV |
| High Positive (% CV) | n = 108 | 6.8% CV |
| In-Transit stability | | |
| 0 hours | n = 60 | 100% concordance |
| 24 hours | n = 60 | 97% concordance |
| 48 hours | n = 60 | 100% concordance |
| 72 hours | n = 60 | 100% concordance |
| 96 hours | n = 60 | 100% concordance |
| 120 hours | n = 60 | 98% concordance |

1. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein i) a portion of the fecal sample is not removed at home or ii) a portion of the fecal sample is not removed by the human subject.

2. The method of claim 1, wherein the fecal sample is a whole fecal sample.

3. The method of claim 1, wherein the portion of the fecal sample is not removed at home.

4. The method of claim 1, wherein the portion of the fecal sample is not removed by the human subject.

5. The method of claim 1, wherein the nucleic acids comprise DNA, RNA, or both.

6. The method of claim 1, wherein the fecal proteins comprise one or more of albumin, hemoglobin, calprotectin, lactoferrin, myeloperoxidase, α1-antitrypsin, lysozyme, elastase, transferrin, immunoglobulin A (IgA), immunoglobulin G (IgG), immunoglobulin M (IgM), pancreatic amylase, trypsin, chymotrypsin, mucin, defensin, or lipocalin-2.

7. The method of claim 1, wherein the fecal sample is collected at home by the human subject via defecation directly into the container.

8. A method of processing a fecal sample, the method comprising:
a) collecting a fecal sample from a human subject, wherein the fecal sample is collected at home by the human subject;
b) combining the fecal sample with a liquid composition in a container, wherein the liquid composition prevents denaturation or degradation of fecal proteins in the fecal sample and wherein the liquid composition maintains integrity of nucleic acids in the fecal sample; and
c) delivering the container containing the fecal sample and the liquid composition to a medical diagnostics laboratory, and
wherein the method does not comprise contacting the fecal sample.

9. The method of claim 8, wherein the method does not comprise contacting the fecal sample by the human subject.

10. The method of claim 8, wherein the method does not comprise contacting the fecal sample with a fecal immunochemical test (FIT).

11. The method of claim 8, wherein the method does not comprise contacting the fecal sample with an immunochemical fecal occult blood test (iFOBT).

12. The method of claim 8, wherein the nucleic acids comprise DNA, RNA, or both.

13. The method of claim 8, wherein the fecal proteins comprise one or more of albumin, hemoglobin, calprotectin, lactoferrin, myeloperoxidase, α1-antitrypsin, lysozyme, elastase, transferrin, immunoglobulin A (IgA), immunoglobulin G (IgG), immunoglobulin M (IgM), pancreatic amylase, trypsin, chymotrypsin, mucin, defensin, or lipocalin-2.

14. The method of claim 8, wherein the fecal sample is collected at home by the human subject via defecation directly into the container.

15. A method of processing a fecal sample, the method comprising:
i) receiving a fecal sample in a liquid composition that prevents denaturation or degradation of one or more fecal proteins in the fecal sample, wherein the liquid composition maintains integrity of one or more human nucleic acids in the fecal sample, and
ii) analyzing the fecal sample for an amount of the fecal protein, and
iii) analyzing the fecal sample for an amount of the human nucleic acid.

16. The method of claim 15, wherein the liquid composition comprises one or more of a buffer, a surfactant, or a ribonuclease inhibitor.

17. The method of claim 15, wherein the nucleic acids comprise DNA, RNA, or both.

18. The method of claim 15, wherein the fecal proteins comprise one or more of albumin, hemoglobin, calprotectin, lactoferrin, myeloperoxidase, α1-antitrypsin, lysozyme, elastase, transferrin, immunoglobulin A (IgA), immunoglobulin G (IgG), immunoglobulin M (IgM), pancreatic amylase, trypsin, chymotrypsin, mucin, defensin, or lipocalin-2.

19. The method of claim 15, wherein the fecal protein is hemoglobin.

20. The method of claim 15, wherein the fecal protein is calprotectin.
